(19) United States
(12) Patent Application Publication
(10) Pub. No.: US 2021/0008118 A1
(43) Pub. Date: Jan. 14, 2021

(54) METHOD FOR ENHANCING MIGRATION OF STEM CELLS INTO CANCER CELLS

(71) Applicant: GACHON UNIVERSITY OF INDUSTRY-ACADEMIC COOPERATION FOUNDATION, Seongnam-si (KR)

(72) Inventors: Dongwoo KHANG, Seoul (KR); Jun-Young PARK, Seoul (KR)

(73) Assignee: GACHON UNIVERSITY OF INDUSTRY-ACADEMIC COOPERATION FOUNDATION, Seongnam-si (KR)

(21) Appl. No.: 16/926,015

(22) Filed: Jul. 10, 2020

(30) Foreign Application Priority Data
Jul. 11, 2019 (KR) 10-2019-0083683

(51) Int. Cl. A61K 35/28; A61K 35/545; C12N 5/0735; C12N 5/0775

(52) U.S. Cl. CPC A61K 35/28; A61K 35/545; C12N 5/0606; A61K 45/06; C12N 5/0664; C12N 5/0665; C12N 2501/999; C12N 5/0663

(57) ABSTRACT

The present invention relates to a method for improving the migration ability of stem cells into cancer cells comprising educating the stem cells by treating the stem cells with an in vitro cell culture medium of cancer cells obtained from a cancer patient to be treated and optionally an anionic channel activator.

### CROSS REFERENCE TO RELATED APPLICATION

This application claims the benefit of Korean Patent Application No. 10-2019-0083683, filed Jul. 11, 2019, contents of which are incorporated herein by reference.

### TECHNICAL FIELD

The present invention is drawn to a method of improving properties of stem cells, more particularly to a method of improving the migration ability of stem cells into cancer cells.

### BACKGROUND OF THE INVENTION

Currently, methods for treating cancer include treatment through surgical operation, radiotherapy, and chemotherapy, but this is accompanied by side effects, or the procedure is limitedly applied depending on the degree of cancer progression. In particular, anticancer drugs used the chemotherapy increased quantitatively as a result of accumulated research, but have not changed significantly in terms of quality. The reason is that most of the anticancer drugs work as a mechanism to stop and kill the cell cycle of actively dividing cells, thereby attacking cells that divide normally besides cancer cells, leading to the side effects of anticancer drugs such as hair loss, loss of appetite, and decrease in immunity due to the reduction of white blood cells. In order to minimize the side effects of these anticancer drugs, the development of targeted anti-cancer drugs is actively taking place, to date, more than 18 targeted anti-cancer drugs have been developed and approved through clinical trials, and more than 200 types of targeted anti-cancer drugs are under clinical trial. However, these targeted anti-cancer drugs have limitations in that they are effective in patients with specific targets, even if they are the same type of cancer, and targeted anti-cancer drugs have problems that cause resistance because they must be administered over a long period of time. Thus, cocktail therapy that uses the targeted anti-cancer drugs in combination with strong anti-cancer drugs and the use of a single anticancer drug to remove cancer in a short time by attacking multiple targets at the same time are considered as alternatives, which also poses a risk of causing serious side effects. Therefore, research on anticancer agents using gold nanoparticles, biodegradable polymers, and carbon nanotubes as targeted anti-cancer agents that can effectively treat cancer with little side effects is actively underway.

In the case of previous nano anti-cancer drugs that have been developed for more effective chemotherapy, biodegradable polymers used in formulations have been easily dissociated from nano-materials under acidic pH and blood conditions, thus limiting the delivery of anti-cancer drugs to target sites and anti-cancer effect of non-degradable nanoparticles such as silica, magnetic nanoparticles and carbon-based nanoparticles at non-toxic doses has not been verified and they have the disadvantages of accumulating in the reticulum endothelial system (RES) organs. These nano-scale anti-cancer drugs still have technical problems to be solved.

As an alternative to this, studies have been attempted to use stem cells having targeting ability against cancer cells as drug delivery vehicles for these nano-scale anti-cancer agents (Zhang et al., Oncotarget, 8(43): 75756-75766, 2017; Auffinger et al., Oncotarget, 4(3): 378-396, 2013; Mooney et al., ACS Nano 8(12): 12450-12460, 2014).

### SUMMARY OF THE INVENTION

However, the above-mentioned prior arts have cost-related problems since the cancer targeting ability of the stem cells is not so high, a large number of stem cells must be used as a drug delivery system.

The present invention is to solve a number of problems, including the problems as described above, an object of the present invention is to provide a method of enhancing the migration ability or targeting ability of stem cells toward cancer cells. However, these problems are exemplary, and the scope of the present invention is not limited thereto.

In an aspect of the present invention, there is provided a method of improving the migration ability of stem cells into cancer cells comprising: educating the stem cells by treating the stem cells with a cell culture medium of in vitro cell culture of cancer cells obtained from a cancer patient to be treated and optionally an anion channel activator.

In another aspect of the present invention, there is provided a composition for improving the migration ability of stem cells into cancer cells comprising an anion channel activator and a cell culture medium of in vitro cell culture of cancer cells obtained from a cancer patient to be treated as an active ingredient.

In another aspect of the present invention, there is provide a drug delivery composition for delivering anti-cancer drugs selectively to cancer cells comprising stem cells educated by treating the stem cells with an anion channel activator and a cell culture medium of in vitro cell culture of cancer cells obtained from a cancer patient to be treated, wherein the stem cells have improved migration ability into the cancer cells of the cancer patient.

In another aspect of the present invention, there is provided a stem cell-nano anti-cancer drug complex in which a carbon nanotube (CNT) or gold nanoparticle (AuNP) loaded with an anti-cancer drug is coupled to the surface of a stem cell, wherein the stem cell is educated in order to improve its migration ability into cancer cells by treating the stem cell with an anion channel activator and a cell culture medium of in vitro cell culture of cancer cells obtained from a cancer patient to be treated.

In another aspect of the present invention, there is provided a pharmaceutical composition for treating cancer comprising the stem cell-nano anti-cancer drug complex as an active ingredient.

In another aspect of the present invention, there is provided a method of treating cancer in a subject in need of treatment comprising administering a stem cell-nano anti-cancer drug complex in which a carbon nanotube (CNT) or gold nanoparticle (AuNP) loaded with an anti-cancer drug is coupled to the surface of a stem cell, wherein the stem cell is educated in order to improve its migration ability into cancer cells by treating the stem cell with an anion channel activator and a cell culture medium of in vitro cell culture of cancer cells obtained from the subject.

In another aspect of the present invention, there is provided a method of treating cancer in a subject in need of treatment comprising:

preparing an educated stem cell by educating a stem cell, wherein the educating is performed by treating the stem cell with cell culture medium of in vitro cell culture of cancer cells obtained from the subject and optionally an anion channel activator to the stem cell;

preparing an educated stem cell-nano anti-cancer drug complex by attaching a nanoparticle loaded with an anti-cancer drug to the educated stem cell; and

administering therapeutically effective amount of the educated stem cell-nano anti-cancer drug complex to the subject

According to one embodiment of the present invention made as described above, since the migration ability of stem cells into targeted cancer cells and the targeting ability is greatly enhanced, even if a smaller amount of stem cells is used, the anticancer treatment effect can be maximized. However, the scope of the present invention is not limited by these effects.

### BRIEF DESCRIPTION OF DRAWINGS

FIG. 1 is a graph showing the experimental results confirming the effect of blocking various chemokines related to the migration of mesenchymal stem cells into cancer cells when the stem cells were co-cultivated with the cancer cells.

FIG. 2A is a schematic diagram representing experimental design for evaluating the migration ability of stem cells into cancer cells by blocking various chemokines or chemokine receptors (CCL1, CCL2, CXCR1 and CXCR4) when the stem cells were co-cultivated with the cancer cells; FIG. 2B is a series of fluorescence microscopic images visualizing adipose-derived mesenchymal stem cells (ADMSCs) stained with a fluorophore migrated into lower layer in which PANC-1 pancreatic cancer cells were located; FIG. 2C is a graph representing the result of FIG. 2B; FIG. 2D is a series of fluorescence microscopic images visualizing bone marrow-derived mesenchymal stem cells (BMMSCs) stained with a fluorophore migrated into lower layer in which A549 lung cancer cells were located when various chemokines or chemokine receptors (CCL2, CXCR1 and CXCR4) were blocked; FIG. 2E is a series of fluorescence microscopic images visualizing bone marrow-derived mesenchymal stem cells (BMMSCs) stained with a fluorophore migrated into lower layer in which H1975 lung cancer cells were located when various chemokines or chemokine receptors (CCL2, CXCR1 and CXCR4) were blocked; FIG. 2F is a series of fluorescence microscopic images visualizing adipose-derived mesenchymal stem cells (ADMSCs) stained with a fluorophore migrated into lower layer in which U87MG brain cancer cells were located when various chemokines or chemok (CCL2, CXCR1 and CXCR4) were blocked; FIG. 2G is a is a graph representing the result of FIG. 2G.

FIG. 3A is a series of fluorescence microscopic images visualizing bone marrow-derived mesenchymal stem cells (BMMSCs) stained with a fluorophore migrated into the lower layer in which lung cancer cells (A549 and H1975) were located, which is an experimental result of analyzing the effect of the anionic channel inhibitor DIDS on the migration of mesenchymal stem cells co-cultivated with the lung cancer cells using a transwell; FIG. 3B is a graph representing the result of FIG. 3A; FIG. 3C is a series of fluorescence microscopic images visualizing aipose-derived mesenchymal stem cells (ADMSCs) stained with a fluorophore migrated into the lower layer in which pancreatic cancer cells (PANC-1) were located, which is an experimental result of analyzing the effect of the anionic channel inhibitor DIDS on the migration of mesenchymal stem cells co-cultivated with the pancreatic cancer cells using a transwell; and FIG. 3D is a graph representing the result of FIG. 3C.

FIG. 4A is a series of fluorescence microscopic images visualizing adipose-derived mesenchymal stem cells (ADMSCs) stained with a fluorophore migrated into the lower layer in which pancreatic cancer cells (PANC-1) were located, which is an experimental result of analyzing the change of migration ability of the stem cells into cancer cells when treating antibodies specific for SLCA4A4 or CLCA4A78 which are sodium bicarbonate cotransporter in order to lower the concentration of proton in the culture medium when the mesenchymal stem cells were co-cultivated with the cancer cells; FIG. 4B is a graph representing the result of FIG. 4A; FIG. 4C is a series of fluorescence microscopic images visualizing adipose-derived mesenchymal stem cells (ADMSCs) stained with a fluorophore migrated into the lower layer in which brain cancer cells (U87MG) were located, which is an experimental result of analyzing the change of migration ability of the stem cells into cancer cells when treating antibodies specific for SLCA4A4 or CLCA4A78 which are sodium bicarbonate cotransporter in order to lower the concentration of proton in the culture medium when the mesenchymal stem cells were co-cultivated with the cancer cells; FIG. 4D is a graph representing the result of FIG. 4C.

FIG. 5A is a series of fluorescence microscopic images visualizing the results of analyzing the migration ability of the stem cells (BMMSCs) into the lower well of the transwell after adjusting pH of the culture medium absent cancer cells (A549) into acidic (pH 6.5), which represents the experimental results determining whether the improvement of the migration ability of the mesenchymal stem cells into cancer cells is caused by the environment of the cancer cells, i.e., low pH; FIG. 5B is a graph representing the experimental results of FIG. 5A; and FIG. 5C is a series of fluorescence microscopic images visualizing the results of analyzing the migration ability of the stem cells (BMMSCs) into the lower well of the transwell after adjusting pH of the culture medium absent cancer cells (PANC-1) into acidic (pH 6.5); FIG. 5D is a graph representing the experimental results of FIG. 5C; and FIG. 5D is a series of fluorescence microscopic images visualizing the results of analyzing the migration ability of the stem cells (ADMSCs) into the lower well of the transwell after adjusting pH of the culture medium absent cancer cells (U87MG) into acidic (pH 6.5); and FIG. 5F is a graph representing the experimental results of FIG. 5E.

FIG. 6A is a series of fluorescence microscopic images visualizing bone marrow-derived stem cells (BMMSCs) into the lower well of the transwell in which lung cancer cells (A549) were located, which is a result of analyzing the degree of migration of the stem cells after treating the stem cells with an anion channel activator forskolin or activating chemokine receptor of the stem cells by treating the stem cells with cell-free culture medium of cancer cells (education) or the forskolin and the cell-free culture medium of cancer cells (education²); FIG. 6B is a graph representing the quantification of the experimental results of FIG. 6A; FIG. 6C is a series of fluorescence microscopic images visualizing adipose-derived stem cells (ADMSCs) into the lower well of the transwell in which pancreatic cancer cells (PANC-1) were located, which is a result of analyzing the degree of migration of the stem cells after treating the stem cells with an anion channel activator, forskolin or activating chemokine receptor of the stem cells by treating cell-free culture medium of cancer cells (education) or treating the stem cells with the forskolin and the cell-free culture medium of cancer cells (education²), and FIG. 6D is a graph representing the quantification of the experimental results of FIG. 6C; FIG. 6D is a graph representing the quantification of the experimental results of FIG. 6C; FIG. 6E is a series of fluorescence microscopic images visualizing adipose-derived stem cells (ADMSCs) into the lower well of the transwell in which brain cancer cells (U87MG) were located, which is a result of analyzing the degree of migration of the stem cells after treating the stem cells with an anion channel activator, forskolin or activating chemokine receptor of the stem cells by treating the stem cells with cell-free culture medium of cancer cells (education) or treating the stem cells with the forskolin and the cell-free culture medium of cancer cells (education²); and FIG. 6F is a graph representing the quantification of the experimental results of FIG. 6E.

FIG. 7 illustrates the experimental results to determine whether the enhancement of tumor targeting effect due to the education of mesenchymal stem cells according to an embodiment of the present invention is a phenomenon specific to cancer cells isolated from a subject; FIG. 7A is a series of fluorescence microscopic images visualizing the migration of bone marrow-derived stem cells (BMMSCs) into the lower well of the transwell in which lung cancer cells (A549) were located, after educating the stem cells with cell-free culture medium of lung cancer cells (A549) or pancreatic cancer cells (PANC-1); FIG. 7B is a graphs representing the result of cell counting of stem cells migrated into the lower well in FIG. 7A; FIG. 7C is a series of fluorescence microscopic images visualizing the migration of adipose-derived stem cells (ADMSCs) into the lower well of the transwell in which pancreatic cancer cells (PANC-1) were located, after educating the stem cells with cell-free culture medium of lung cancer cells (A549) or pancreatic cancer cells (PANC-1); FIG. 7D is a graphs representing the result of cell counting of stem cells migrated into the lower well in FIG. 7C.

FIG. 8A is a photograph taken by a whole body in vivo fluorescence imaging in xenograft model animals prepared by the inoculation of lung cancer cells (A549) and administered with the educated bone marrow-derived mesenchymal stem cells (BMMSCs) according to an embodiment of the present invention which visualizes the distribution of the stem cells in the model animals; FIG. 8B is a series of photographs showing results obtained by in vivo fluorescence imaging which shows the distribution of mesenchymal stem cells in main organs and tumor tissues excised from the experimental animals of FIG. 8A; and FIG. 8C is a series of histograms showing the results of flow cytometry analysis on markers specific to lung cancer cells and bone marrow-derived mesenchymal stem cells after dissociating the lung tissue excised from the experimental animals into single cells.

FIG. 9A is a photograph taken by a whole body in vivo fluorescence imaging in xenograft model animals prepared by inoculating pancreatic cancer cells (PANC-1) and administered with the educated adipose-derived mesenchymal stem cells (ADMSCs) according to an embodiment of the present invention which visualizes the distribution of the stem cells in the model animals; FIG. 9B is a series of photographs showing results obtained by the in vivo fluorescence imaging which shows the distribution of mesenchymal stem cells in main organs and tumor tissues excised from the experimental animals of FIG. 9A; and FIG. 9C is a series of histograms showing the results of flow cytometry analysis on markers specific to pancreatic cancer cells and adipose-derived mesenchymal stem cells after dissociating the pancreatic tissue excised from the experimental animals into single cells.

FIG. 10A is a photograph taken by a whole body in vivo fluorescence imaging in xenograft model animals prepared by the inoculation of brain cancer cells (U87MG) and administered with the educated adipose-derived mesenchymal stem cells (ADMSCs) according to an embodiment of the present invention which visualizes the distribution of the stem cells in the model animals; FIG. 10B is a series of photographs showing results obtained by the in vivo fluorescence imaging which shows the distribution of mesenchymal stem cells in main organs and tumor tissues excised from the experimental animals of FIG. 10A; and FIG. 10C is a series of histograms showing the results of flow cytometry analysis on markers specific to brain cancer cells and adipose-derived mesenchymal stem cells after dissociating the brain tissue excised from the experimental animals into single cells.

### DETAILED DESCRIPTION OF THE INVENTION

### Definition of Terms

The term “mesenchymal stem cell (MSC)” as used herein means adult mesenchymal stem cell (MSC) among adult stem cells and is derived from bone marrow, umbilical cord blood, and adipocytes. It is characterized by having multipotency.

The term “nano anti-cancer drug complex” as used herein means a drug delivery system for anti-cancer agents using nanotechnology which is used for controlling release and absorption of anticancer drugs, or targeting and delivering anticancer drugs to specific sites of the body, and maximizing efficacy of anti-cancer drugs while reducing side effects thereof as well as retention of anti-cancer drugs for a certain period of time in the targeted site.

The term “immune checkpoint inhibitor” as used herein refers to a drug that blocks certain types of immune system cells, such as T lymphocytes, and certain proteins produced by some cancer cells. These proteins inhibit immune responses and prevent T lymphocytes from killing cancer cells. Therefore, when these proteins are blocked, the immune system's “braking system” is released and T lymphocytes can kill cancer cells better. PD-1/PD-L1 and CTLA-4/B7-1/B7-2 are well known so far as the “immune checkpoint”. Examples of PD-1 inhibitors include Pembrolizumab (trade name: Keytruda), Nivolumab (trade name: Opdivo), and the PD-1 ligand, PD-L1 inhibitor, Atezolizumab (trade name: Tecentriq), and Avelumab (trade name: Bavencio) Etc. are present. Meanwhile, as a CTLA-4 inhibitor that inhibits the interaction of CTLA-4/B7-1/B7-2, Ipilimumab (trade name: Yervoy) has been approved by the USFDA. In recent years, it has been impressively successful, especially in patients with metastatic melanoma or Hodgkin's lymphoma, and has shown great potential in clinical trials in other types of cancer patients.

The term “antibody” as used herein also referred as an immunoglobulin, refers to a Y-shaped protein produced from plasma cells which is used by the immune system to identify or neutralize foreign substances such as bacteria and viruses. The antibodies used herein include various “functional fragments” derived from antibodies, such as Fab, F(ab′)₂, Fab′, scFv and sdAb.

The term “functional fragment of an antibody” as used herein means a fragment derived from the antibody, which retains antigen-binding activity, and includes both a fragment produced by cutting the antibody with an endopeptidase as well as a single-chain fragment produced by a recombinant method.

The term “Fab” as used in this document is an antigen-binding antibody fragment (fragment antigen-binding), a fragment produced by cutting an antibody molecule with a protease, papain, a dimer of two peptides, V_{H}—CH1 and V_{L}—CL, and another fragment produced by papain is referred to as Fc (fragment crystallizable).

The term “F(ab′)₂” as used in this document is a fragment containing an antigen-binding site among fragments produced by cleaving an antibody with a protease, pepsin, and refers to a tetrameric form in which the two Fabs are connected by disulfide bonds. Another fragment produced by pepsin is referred to as pFc′.

The term “Fab′” as used herein refers a molecule having a structure similar to Fab produced by separating the F(ab′)₂ under weak reducing conditions.

The term “scFv” as used herein is an abbreviation of “single chain variable fragment” and is not a fragment of an actual antibody. It is produced by linking heavy chain variable region (V_{H}) and light chain variable region (V_{L}) with a linker peptide of about 25 a.a. It is known to possess antigen binding ability even though it is not a unique antibody fragment (Glockshuber et al., Biochem. 29(6): 1362-1367, 1990).

The term “sdAb (single domain antibody)” as used in this document is referred to as a nanobody, and is an antibody fragment composed of a single variable region fragment of an antibody. The sdAb derived from the heavy chain is mainly used, but a single variable region fragment derived from the light chain is also reported to be a specific binding to the antigen.

The term “antibody mimetic” as used herein refers to a single chain antibody fragment derived from camelids or cartilaginous (V_{H}H or V_{NAR}) which consists of only a heavy chain except a light chain or an antibody-like protein prepared from non-antibody scaffold protein such as Alphabody, Avimer, Affilin, nanoCLAMPs, Adnectin, Affibody, Anticalin, DARPin, Fynomer, Kunitz domain, monobody, and variable lymphocyte receptors (VLRs).

### MODE FOR THE INVENTION

In an aspect of the present invention, there is provided a method of improving the migration ability of stem cells into cancer cells comprising: educating the stem cells by treating the stem cells with a cell culture medium of in vitro cell culture of cancer cells obtained from a cancer patient to be treated and optionally an anion channel activator.

In the above method, the stem cells may be embryonic stem cells or mesenchymal stem cells, and the mesenchymal stem cells can be bone marrow-derived mesenchymal stem cells, adipose-derived mesenchymal stem cells, umbilical cord-derived mesenchymal stem cells, dental pulp-derived stem cells or peripheral blood-derived stem cells.

In the above method, the anion channel activator may be a Cl⁻ channel activator or a bicarbonate channel activator, and the anion channel activator may be forskolin, denufosol, brevenal, lubiprostone, N-aroylaminothiazole “activators” (E_{act}) analogue. These anionic channel activators are well documented in Namkung et al. (FASEB J. 25(11): 4048-4062, 2011). The above document is incorporated herein by reference.

In another aspect of the present invention, there is provided a composition for improving the migration ability of stem cells into cancer cells comprising an anion channel activator and a cell culture medium of in vitro cell culture of cancer cells obtained from a cancer patient to be treated as an active ingredient.

In the composition, the stem cells may be embryonic stem cells or mesenchymal stem cells, and the mesenchymal stem cells are bone marrow-derived mesenchymal stem cells, adipose-derived mesenchymal stem cells, umbilical cord-derived mesenchymal stem cells, dental pulp-derived stem cells or peripheral blood-derived stem cells.

In the composition, the anion channel activator may be a Cl⁻ channel activator or a bicarbonate channel activator, and the anion channel activator may be forskolin, denufosol, brevenal, lubiprostone, N-aroylaminothiazole “activators” (E_{act}) analogue.

In another aspect of the present invention, there is provide a drug delivery composition for delivering anti-cancer drugs selectively to cancer cells comprising stem cells educated by treating the stem cells with an anion channel activator and a cell culture medium of in vitro cell culture of cancer cells obtained from a cancer patient to be treated, wherein the stem cells have improved migration ability into the cancer cells of the cancer patient.

In another aspect of the present invention, there is provided a stem cell-nano anti-cancer drug complex in which a carbon nanotube (CNT) or gold nanoparticle (AuNP) loaded with an anti-cancer drug is coupled to the surface of a stem cell, wherein the stem cell is educated in order to improve its migration ability into cancer cells by treating the stem cell with an anion channel activator and a cell culture medium of in vitro cell culture of cancer cells obtained from a cancer patient to be treated.

In the stem cell-nano anti-cancer drug complex, the anticancer drug may be a chemical agent or a biologic agent, and the chemical agent may be doxorubicin, paclitaxel, ABT737, 5-fluorouracil, BCNU, CCNU, 6-mercaptopurine, nitrogen Mustard, cyclophosphamide, vincristine, vinblastine, cisplatin, mesotrexate, cytarabine thiotepa, busulfan or procarbazine. In addition, the biologic agent may be an immune checkpoint inhibitor, immune activating protein or an antibody or a functional fragment thereof targeting a cancer marker protein. In the stem cell-nano anti-cancer drug complex, the immune checkpoint may be PD-1, PD-L1, CTLA-4, B7-1 or B7-2, and the immune checkpoint inhibitor may an inhibitor of PD-1/PD-L1 interaction or an inhibitor of CTLA-4/B7-1/B7-2 interaction. The inhibitor of PD-1/PDL1 interaction may be an antibody targeting PD-1 or PD-L1, a functional fragment of the antibody, or a single chain-based antibody mimetic, and the inhibitor of CTLA-4/B7-1/B7-2 interaction may be an antibody targeting the CTLA-4, B7-1 or B7-2, a functional fragment of the antibody, or a single chain-based antibody mimetic, and the antibody targeting the PD-1 or PD-L1 may be Pembrolizumab, Nivolumab, Atezolizumab or Avelumab, and the inhibitor of CTLA-4/B7-1/B7-2 interaction may be ipilimumab. The immune-activating protein may be C-reactive protein, serum amyloid P component, serum amyloid A, mannan-binding lectin, fibrinogen, prothrombin, factor VIII, von Willebrand factor, plasminogen activator inhibitor-1 (PAI-1), alpha 2-macroglobulin, hepcidin, ceruloplasmin, haptoglobin, orosomucoid (alpha-1-acid glycoprotein, AGP), alpha 1-antitrypsin, or alpha 1-antichymotrypsin. The cancer marker protein, an overexpressed protein on the surface of cancer cells, may be AFP (alphafetoprotein), epidermal growth factor receptor (EGFR), carcinoembryonic antigen (CEA), CA-123, MUC-1, epithelial tumor antigen (ETA), tyrosinase, or MAGE (melanoma-associated antigen).

In the stem cell-nano anti-cancer drug complex, the nanoparticle may be an organic nanoparticle or an inorganic nanoparticle, and the organic nanoparticle may be a porous or shell/core structure composed of a biodegradable polymer. The biodegradable polymer may be PLGA{poly(lactic-co-glycolic acid)}, PVA{poly(vinyl alcohol)}, PGA{poly(glycolic acid)}, PLA{poly(lactic acid)}, PCL{poly(caprolactone)}, PHA{poly(hydroxyalkanoate)}, aliphatic polyester, or mixtures thereof. In addition, the inorganic nanoparticles may be gold nanoparticles or carbon nanotube-based nanoparticles.

In the stem cell-nano anti-cancer drug complex, the nanoparticle may be attached to the stem cell by attaching an antibody specific to the stem cell marker protein, wherein the antibody is attached on the surface of the nanoparticle and the stem cell marker protein may be CD90, CD73 or CD105.

In the stem cell-nano anti-cancer drug complex, the nanoparticles may be coated with a polymer material having a carboxyl group, and the polymer material having a carboxyl group is carboxylated PEG (polyethylene glycol), hyaluronic acid (PHA), polyhydroxyalkanoates (PHA), PLGA{poly(lactic-co-glycolic acid)}, PLA{poly(lactic acid)} or PGA{poly(glycolic acid)}.

Alternatively, when the anti-cancer drug is a biologic agent, the stem cells may be transformed to directly express the biologic agent. In this case, a gene construct comprising a polynucleotide encoding the biologic agent operably linked to a transcription regulator suitable for gene expression of a stem cell, such as a promoter and an enhancer may be prepared and then transduced with the stem cells in various ways for preparing transduced stem cells.

In another aspect of the another aspect of the present invention, there is provided a pharmaceutical composition for treating cancer comprising the stem cell-nano anti-cancer drug complex as an active ingredient.

The pharmaceutical composition for treating cancer of the present invention may include a pharmaceutically acceptable carrier. The composition comprising a pharmaceutically acceptable carrier may be various oral or parenteral formulations, but is preferably a parenteral formulation. In the case of formulation, it is prepared using diluents or excipients such as fillers, extenders, binders, wetting agents, disintegrating agents and surfactants. Solid preparations for oral administration include tablets, pills, powders, granules, and capsules, etc. These solid preparations are prepared by mixing at least one excipient such as starch, calcium carbonate, sucrose or lactose, gelatin, etc. with the active ingredient. In addition, lubricants such as magnesium stearate and talc may be used in addition to simple excipients. Liquid preparations for oral administration include suspending agents, oral liquid solutions, emulsions, syrups, etc. In addition to water and liquid paraffin, which are commonly used as diluents, various excipients such as wetting agents, sweeteners, fragrances, and preservatives can be included. Formulations for parenteral administration include sterile aqueous solutions, non-aqueous solvents, suspensions, emulsions, lyophilized preparations, and suppositories. As the non-aqueous solvent and suspension solvent, propylene glycol, polyethylene glycol, vegetable oil such as olive oil, and injectable ester such as ethyl oleate may be used. As a base for suppositories, witepsol, macrogol, tween 61, cacao butter, laurin butter, and glycerogelatin may be used.

The pharmaceutical composition for treating cancer of the present invention may be any selected preparations from the group consisting of tablets, pills, powders, granules, capsules, suspensions, solutions, emulsions, syrups, sterilized aqueous solutions, non-aqueous solvents, suspensions, emulsions, lyophilizers and suppositories.

The pharmaceutical composition for treating cancer of the present invention may be administered orally or parenterally, and when administered parenterally, it can be administered through various routes such as intravenous injection, intranasal inhalation, intramuscular administration, intraperitoneal administration, and percutaneous absorption.

The pharmaceutical composition for treating cancer of the present invention may be administered in a therapeutically effective amount.

The term “therapeutically effective amount” as used herein refers to an amount sufficient to treat the disease at a reasonable benefit/risk ratio applicable to medical treatment, and the effective dose level may be determined according to factors including the type of subject, severity of disease, age, sex, and activity of anticancer agents, sensitivity to anticancer agents, time of administration, route of administration and rate of excretion, duration of treatment, concurrent anticancer agents, and other factors well known in the art. The pharmaceutical composition of the present invention may be administered at a dose of 0.1 mg/kg to 1 g/kg, more preferably 1 mg/kg to 500 mg/kg. Meanwhile, the dosage may be appropriately adjusted according to the patient's age, gender and condition.

The pharmaceutical composition for treating cancer according to the present invention may be administered as an individual therapeutic agent or in combination with other anti-cancer agents, and may be administered sequentially or simultaneously with other conventional anti-cancer agents. And it can be administered as single or multiple administration. Considering all of the above factors, it is important to administer an amount that can achieve the maximum effect in a minimal amount without side effects, and can be easily determined by those skilled in the art.

In another aspect of the present invention, there is provided a method of treating cancer in a subject in need of treatment comprising administering a stem cell-nano anti-cancer drug complex in which a carbon nanotube (CNT) or gold nanoparticle (AuNP) loaded with an anti-cancer drug is coupled to the surface of a stem cell, wherein the stem cell is educated in order to improve its migration ability into cancer cells by treating the stem cell with an anion channel activator and a cell culture medium of in vitro cell culture of cancer cells obtained from the subject.

In another aspect of the present invention, there is provided a method of treating cancer in a subject in need of treatment comprising:

preparing an educated stem cell by educating a stem cell, wherein the educating is performed by treating the stem cell with cell culture medium of in vitro cell culture of cancer cells obtained from the subject and optionally an anion channel activator to the stem cell;

preparing an educated stem cell-nano anti-cancer drug complex by attaching a nanoparticle loaded with an anti-cancer drug to the educated stem cell; and

administering therapeutically effective amount of the educated stem cell-nano anti-cancer drug complex to the subject.

The present inventors focus on the ability of stem cells with targeting ability to cancer cells and prepared stem cell-anti-cancer loaded nanoparticle complexes in which nanoparticles, such as gold nanoparticles, which are loaded with anticancer compounds are attached on the surface of stem cells and confirmed safety and therapeutic effect thereof. However, since the cancer targeting ability of stem cells is not complete, the stem cell anti-cancer drug complex has a problem that requires a large amount of stem cells. This is a big obstacle in the development of anti-cancer therapeutics using stem cells, because it takes a great cost to cultivate and proliferate stem cells. Accordingly, there is a need to enhance the targeting ability of stem cells to cancer cells so that the maximum effect can be achieved even in a smaller amount.

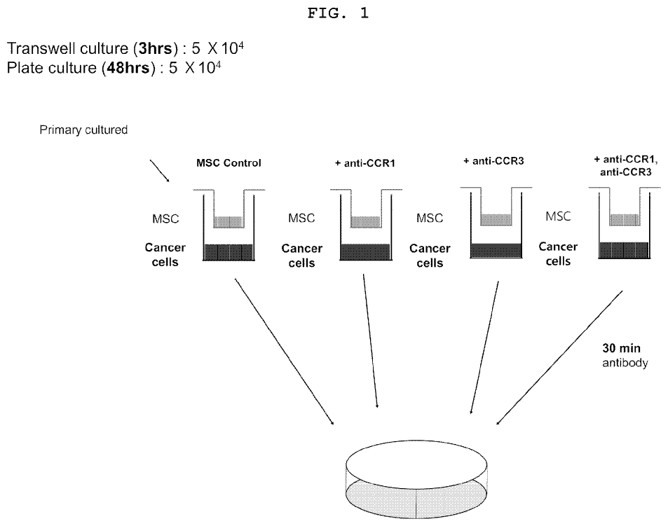

Thus, the present inventors tried to maximize the targeting ability of stem cells to cancer cells. As a result, the present inventors confirmed that when activating the anion channels of the stem cells, the ability of the stem cells to migrate into the target cancer cells is enhanced when the stem cells are educated by treating cell culture medium of cancer cells isolated from a cancer patient to be treated for a certain period of time, and when the stem cells are educated by treating both the components (anion channel activator and cell culture medium of the target cancer cells), the migration potency of the stem cells into target cancer cells can be elevated significantly (FIG. 1).

Hereinafter, the invention will be described in detail through examples. However, the present invention is not limited to the embodiments disclosed below, but can be implemented in various different forms, and the following embodiments make the disclosure of the present invention complete, and inform the scope of the invention to those skilled in the art completely.

### EXAMPLES

### Example 1: Measurement of In Vitro Mobility of Stem Cells

In vitro mobility measurement of stem cells according to an embodiment of the present invention was performed using a transwell membrane bilayer plate having a pore size of 8 μm. Cancer cells for stimulating the stem cells were cultured in the layer under the membrane, and the stem cells were cultured in the upper chamber to allow the cells to penetrate downward. At this time, the mobility was measured through the number of stem cells infiltrated the membrane.

1-1: Search for Stem Cell Tracking Ability According to Chemokine Derived from Cancer Cells

The present inventors sought to investigate whether the ability of stem cells to migrate into cancer cells is due to a response to chemokine secreted by cancer cells. To this end, the present inventors investigated the influence of chemokines by inhibiting chemokine receptors of stem cells that respond to chemokines secreted from cancer cells with antibodies (FIG. 1). Specifically, in the case of lung cancer cells, 5×10⁴ of the bone marrow-derived mesenchymal stem cells (BMMSCs) as a control and the lung cancer cells (A549 and H1975) as experimental groups were dispensed onto the lower well of the transwells, respectively, and cultivated for 24 hours at 37° C. Thereafter, 5×10⁴ BMMSCs treated with antibodies specifically binding to chemokine receptors (CXCR1, CXCR4, and CCL2) for 30 minutes were dispensed onto the transwells and the transwells were inserted into the lower wells, and incubated at 37° C. for 6 hours. In the case of pancreatic cancer cells, 5×10⁴ each of the adipose-derived mesenchymal stem cells (ADMSCs) as a control group and pancreatic cancer cells (PANC-1) as an experimental group were dispensed onto the lower layer of the transwells and incubated at 37° C. for 48 hours. Thereafter, each 5×10⁴ of ADMSCs treated with antibodies specifically binding to the chemokine receptors (CCR1 and CCR3) for 30 minutes were dispensed onto transwells, and the transwells were inserted into new lower wells in which no cancer cells are present, and cultured at 37° C. for 3 hours. In the case of brain tumor cells, each 5×10⁴ of adipose-derived mesenchymal stem cells (ADMSCs) as a control group and brain cancer cells (U87MG) as an experimental group were cultured at 37° C. for 48 hours. Then, the ADMSCs were treated with the antibodies specifically binding to the chemokine receptors (CCR1, CCR5 and CXCR4) for 30 minutes, and then ADMSCs were counted and each 5×10⁴ of ADMSCs were seeded in the transwells and incubated at 37° C. for 3 hours. After incubation, the cells migrated into the lower well were fixed with methanol, and the cell nuclei were stained with DAPI (4′,6-diamidino-2-phenylindole). Subsequently, stem cells migrated were counted through a confocal fluorescence microscope in order to measure their mobility.

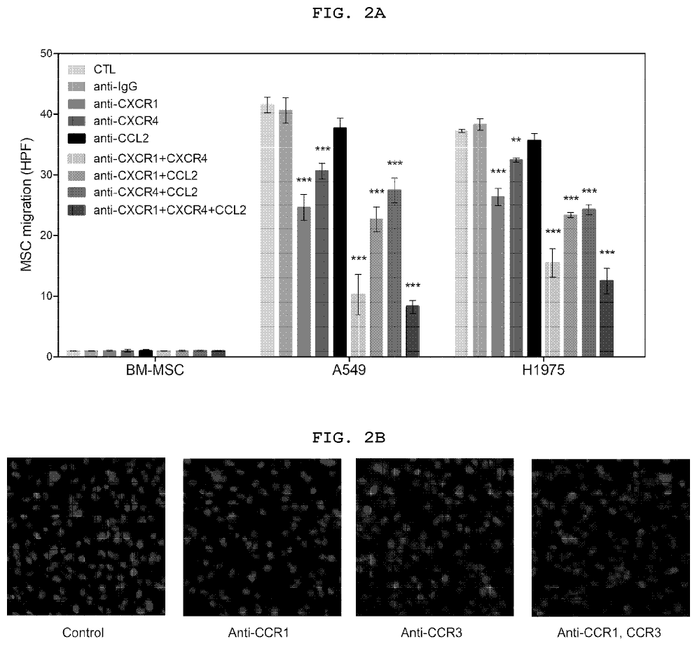

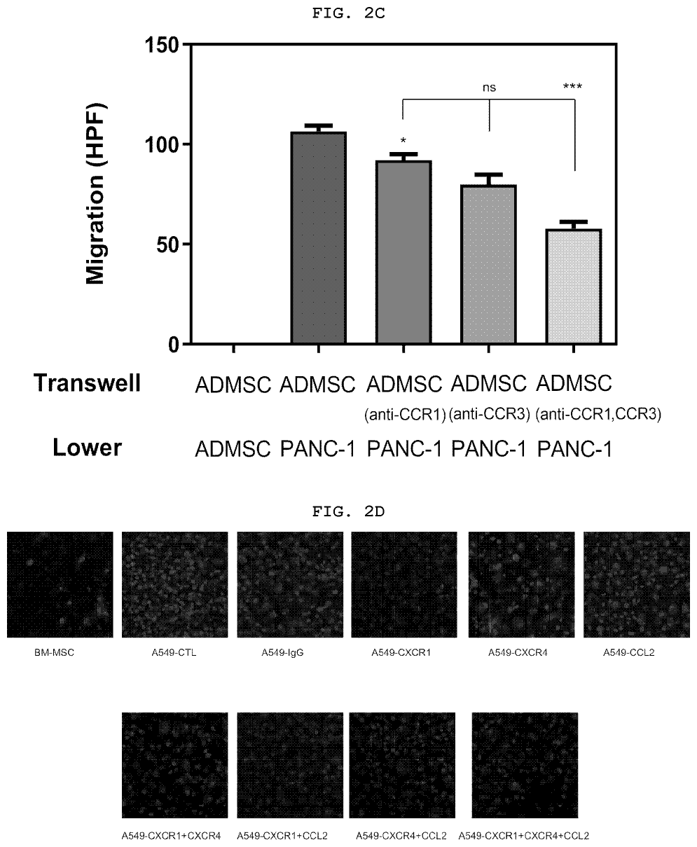

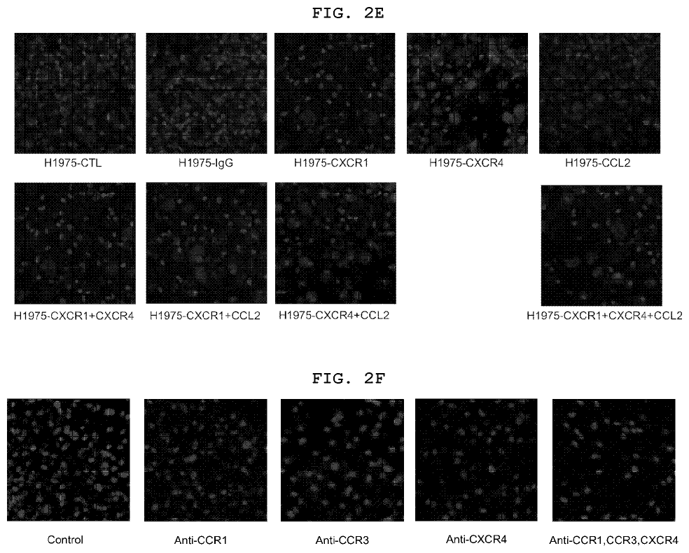

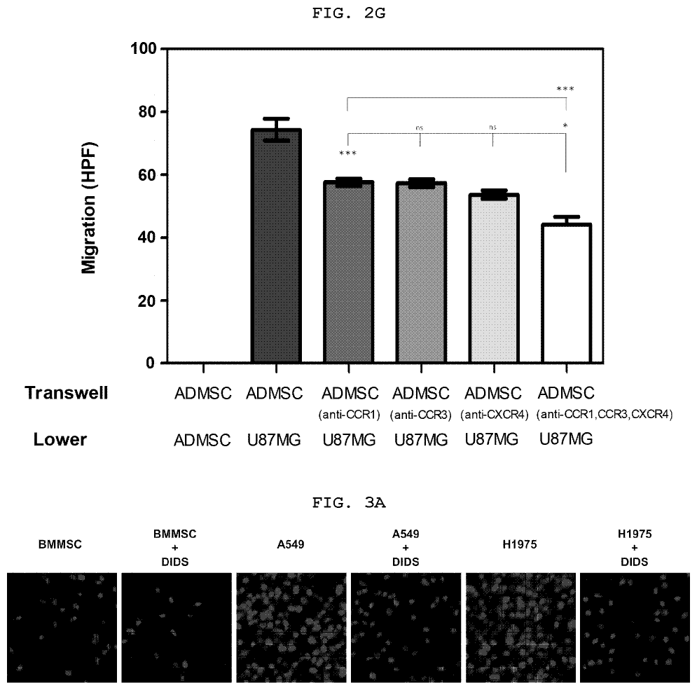

As a result, as shown in FIGS. 2A to 2G, the ability of stem cells to migrate into cancer cells has been shown to decrease by blocking chemokines or chemokine receptors, particularly when blocking more than one chemokine or chemokine receptor simultaneously the mobility of stem cells was decreased dramatically. This suggests that the recognition of the chemokine secreted by cancer cells by the chemokine receptor present on the surface of the stem cells is an important reaction in the chemotaxis of stem cells to cancer cells.

### 1-2: Investigation of Stem Cell Migration Ability According to Ion Channel Activity

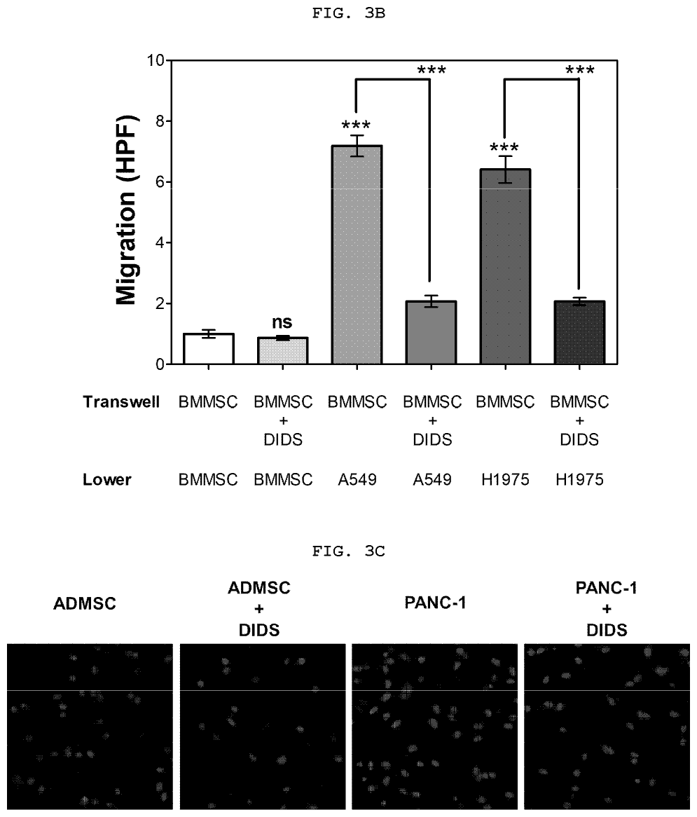

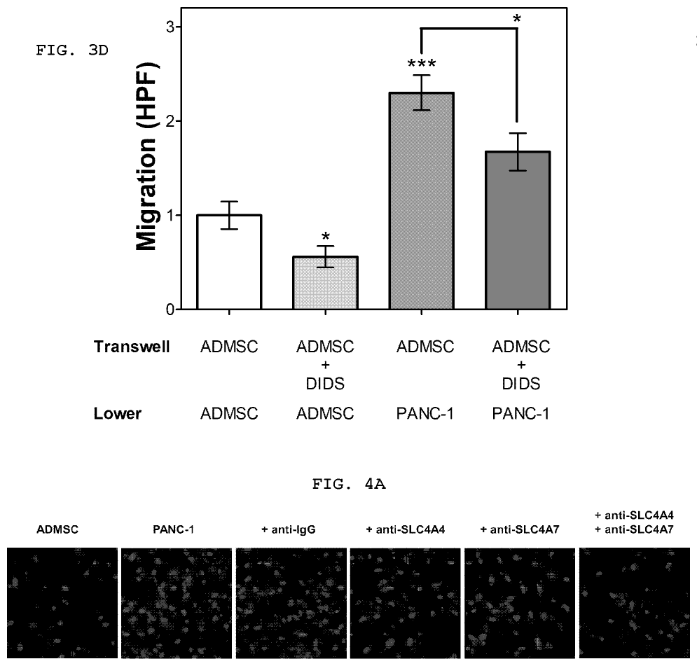

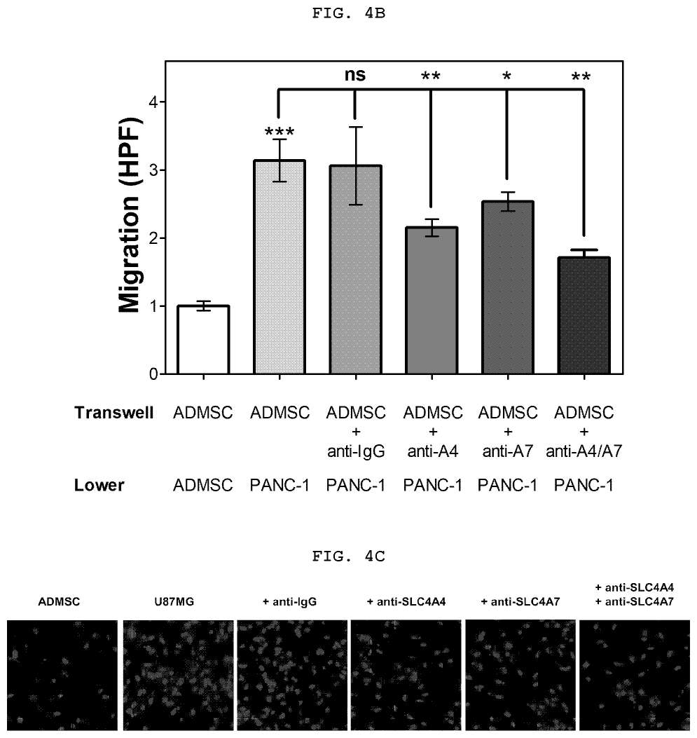

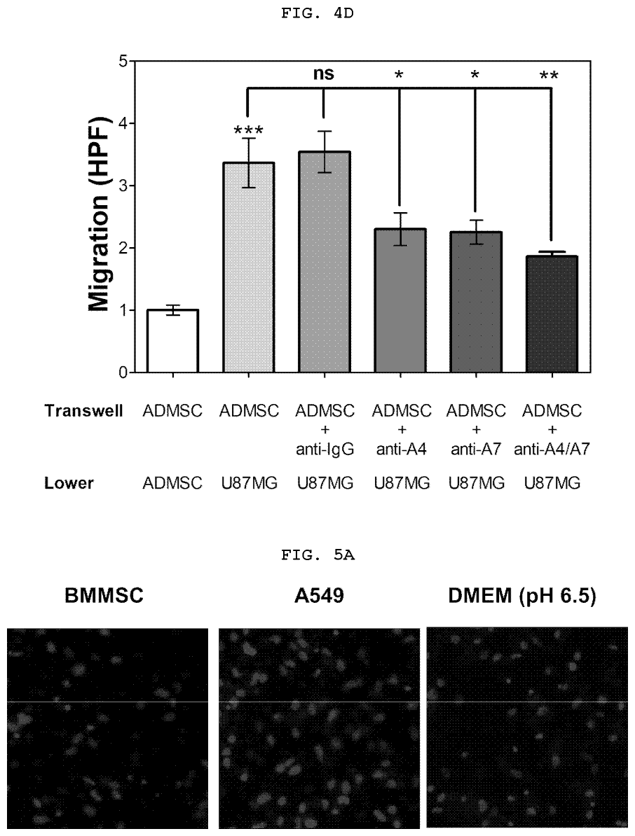

In order to investigate the effect of ion channel activity on the ability of stem cells to migrate into cancer cells, the present inventors performed an analysis similar to Example 1-1 using DIDS (4,4′-Diisothiocyano-2,2′-stilbenedisulfonic acid) which is an ion channel inhibitor and antibodies specifically binding to ion channels (SLC4A4, SLC4A7). Particularly, in the case of lung cancer cells, 5×10⁴ of bone marrow-derived mesenchymal stem cells (BMMSCs) as a control group and lung cancer cells (A549 and H1975) as an experimental group were dispensed onto the lower well, respectively, and cultured at 37° C. for 24 hours. Thereafter, 5×10⁴ of BMMSCs treated with 500 μM of DIDS or antibodies specifically binding to the ion channel were dispensed onto the transwell, and then cultured at 37° C. for 6 hours. In the case of pancreatic cancer cells, each 5×10⁴ cells of ADMSCs as a control group and pancreatic cancer cells (PANC-1) as an experimental group were dispensed onto the lower well and incubated at 37° C. for 48 hours. Thereafter, 5×10⁴ cells of ADMSCs treated with DIDS or antibodies specifically binding to ion channels were seeded on the transwells, and the transwells were inserted into the lower wells, and further cultured for 3 hours. In the case of brain tumor cells, each 5×10⁴ cells of ADMSCs as a control group and brain tumor cells (U87MG) as an experimental group were dispensed onto the lower well and incubated at 37° C. for 48 hours. Thereafter, 5×10⁴ ADMSCs treated with DIDS or antibodies specifically binding to ion channels were seeded on the transwells, and the transwells were inserted into the lower wells, and further cultured for 3 hours. After the cultivation, the cells of the lower layer wells were fixed with methanol, and the nuclei of stem cells migrated into the lower well were stained with DAPI (4′,6-diamidino-2-phenylindole). Subsequently, stem cells migrated into the lower well through the transwell membrane were counted trough a confocal fluorescence microscope in order to measure their mobility (FIGS. 3A to 4D).

As a result, as shown in FIGS. 3A to 4D, the ability of the mesenchymal stem cells to migrate into all cancer cells used in the experiment is decreased significantly by treating anion channel blocker DIDS and antibodies specific for ion channels (anti-SLC4A4, anti-SLC4A7). This suggests that the activity of the anion is an important factor in the mobility of stem cells into cancer cells.

### 1-3: Effect of Acidity on Stem Cell Migration

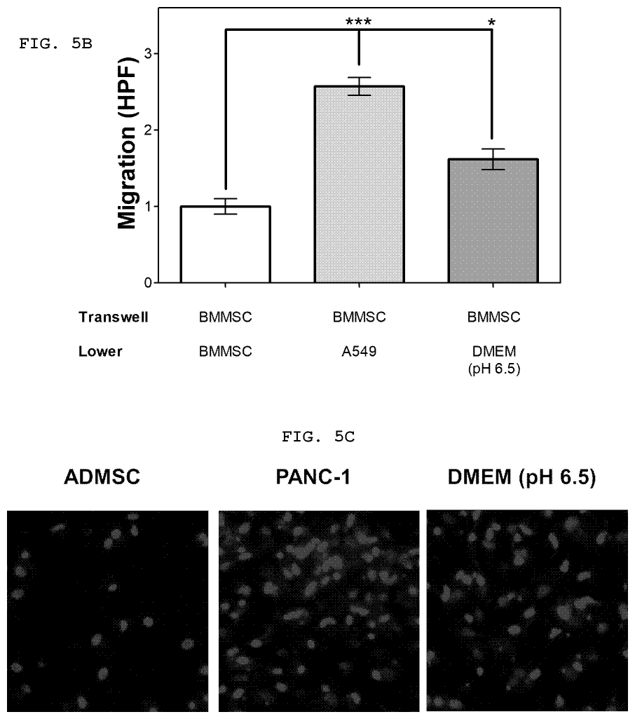

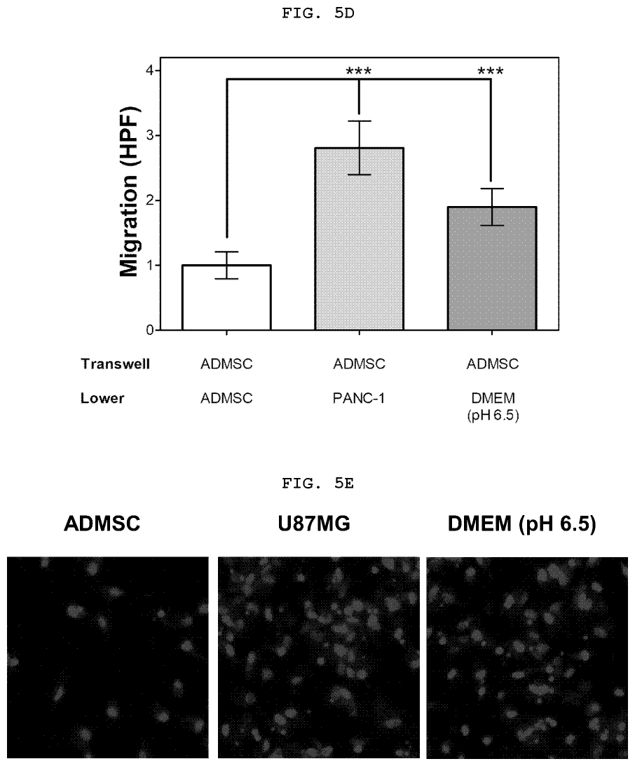

The microenvironment of cancer cells is acidic, unlike normal tissue. Accordingly, the present inventors tried to investigate the effect of the microenvironment, that is, the acidic condition of cancer, on the migration of stem cells into the cancer cells. To this end, specifically, 5×10⁴ of bone marrow-derived mesenchymal stem cells (BMMSCs) as a control group and lung cancer cells (A549) as an experimental group, respectively, were dispensed onto the lower well, and the culture medium adjusted to pH 6.5 without cancer cells was dispensed onto the experimental group, and then the cells were cultivated for 24 hours at 37° C. Then, 5×10⁴ BMMSCs were dispensed onto the transwells, and the trasnwells were inserted into the lower wells, and cultured for 6 hours. In the case of pancreatic cancer cells, 5×10⁴ of adipose-derived mesenchymal stem cells (ADMSCs) as a control group and pancreatic cancer cells (PANC-1) as an experimental group were dispensed onto the lower wells, respectively, and the culture medium adjusted to pH 6.5 without cancer cells was dispense into the experimental group and the cells were further incubated at 37° C. for 24 hours. And then, 5×10⁴ cells of ADMSCs were seeded into the transwells, and the transwells were inserted into the lower wells, and cultured for 3 hours. In the case of brain cancer cells, 5×10⁴ of adipose-derived mesenchymal stem cells (ADMSCs) as a control group and brain cancer cells (U87MG) as an experimental group were dispensed onto the lower wells, respectively, and the culture medium adjusted to pH 6.5 without cancer cells was dispense into the experimental group and the cells were further incubated at 37° C. for 24 hours. After culturing, the cells of the lower layer wells were fixed with methanol, and the nuclei of stem cells stained with DAPI (4′,6-diamidino-2-phenylindole). Subsequently, stem cells migrated into the lower chamber were counted through a confocal fluorescence microscope in order to measure their mobility (FIGS. 5A to 5F).

As a result, as shown in FIGS. 5A to 5F, it was confirmed that even in the absence of cancer cells, the migration of stem cells into the medium with pH adjusted to mild acidic value was significantly increased compared to the control. This suggests that the stem cells exhibit a pH-dependent mobility in a mechanism independent of the reaction with chemokines secreted from cancer cells.

### 1-4: Analysis of the Effect of Education on Stem Cells

The present inventors investigated whether the ability of stem cells to migrate into cancer cells can be enhanced when educating stem cells from the results of the above-described Examples 1-1 to 1-3. Particularly, in the case of lung cancer cells, the present inventors dispense 5×10⁴ each of bone marrow-derived mesenchymal stem cells (BMMSCs) as a control and lung cancer cells (A549 and H1975) as experimental groups were dispensed onto the lower well, and cultivated for 24 hours at 37° C. Simultaneously with the cultivation of the lower well, the BMMSCs were inoculated into a cell-free medium in which lung cancer cells had been cultured at 37° C. for 24 hours, and then cultured at 37° C. for 24 hours, followed by dispensing 5×10⁴ cells onto the transwells. The transwells were inserted into the lower wells and further cultured for 6 hours (education). In the case of pancreatic cancer cells, 5×10⁴ cells of adipose-derived mesenchymal stem cells (ADMSCs) as a control group and pancreatic cancer cells (PANC-1) as a control group were dispensed onto the lower wells and cultured at 37° C. for 48 hours. Simultaneously with the culture of the lower well, like the lung cancer cells, ADMSCs were inoculated with cell-free medium in which pancreatic cancer cells had been cultured at 37° C. for 24 hours, and further cultured at 37° C. for 24 hours, followed by dispensing 5×10⁴ cells in the transwells. The transwells were inserted to the lower wells and the cells were further cultivated for 3 hours (education). In the case of brain cancer cells, 5×10⁴ cells of adipose-derived mesenchymal stem cells (ADMSCs) as a control group and brain cancer cells (U87MG) as a control group were dispensed onto the lower wells and cultured at 37° C. for 48 hours. Simultaneously with the culture of the lower well, like the lung cancer cells and the pancreatic cancer cells, ADMSCs were inoculated with cell-free medium in which pancreatic cancer cells had been cultured at 37° C. for 48 hours, and further cultured at 37° C. for 24 hours, followed by dispensing 5×10⁴ cells onto the transwells. The transwells were inserted to the lower wells and the cells were further cultured for 3 hours (education). On the other hand, in Example 1-3, from the result that the ability of stem cells to migrate into cancer cells was decrease when anion channel inhibitor was treated, the present inventors sought to investigate whether the ability of stem cells to migrate into cancer cells is enhanced when anion channel is activated. To this end, the present inventors treated 100 nM forskolin which is an anion channel activator anole (+ forskolin) or in combination with the cell-free culture medium in which cancer cells had been cultivated (education² or e²) to the stem cells in all experiments.

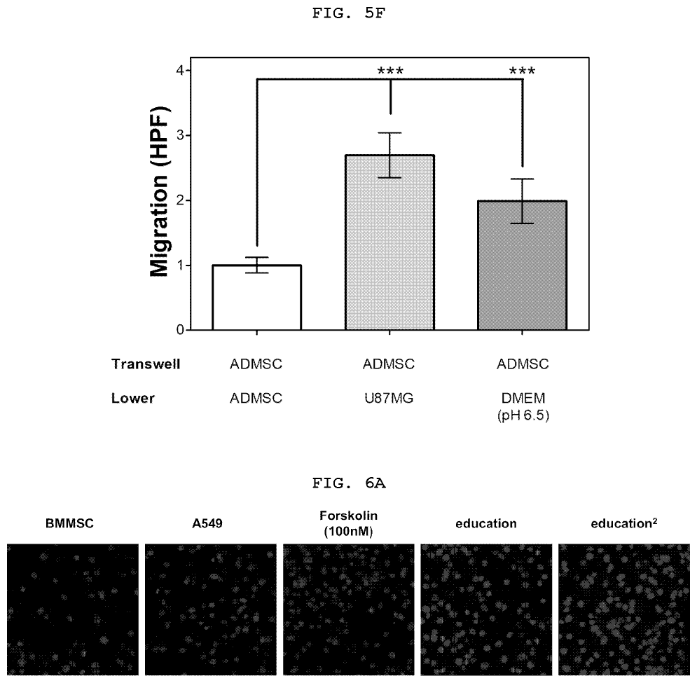

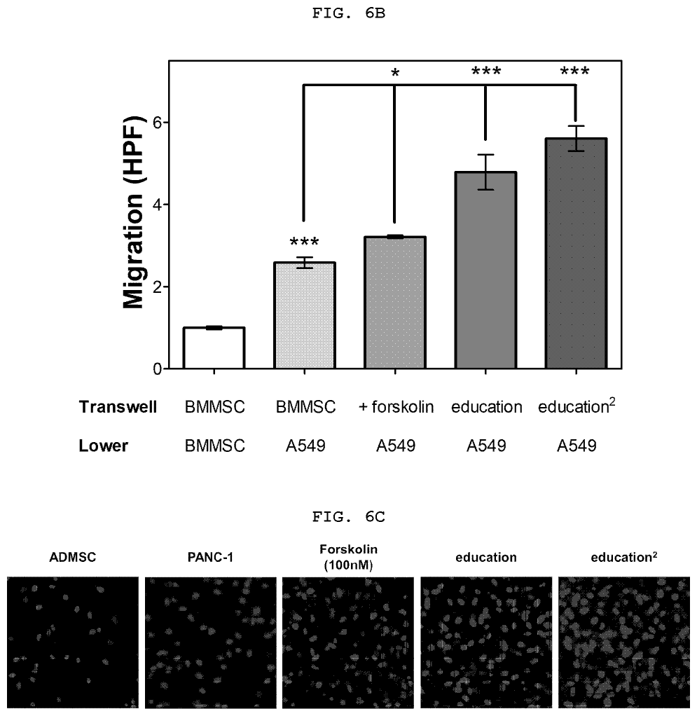

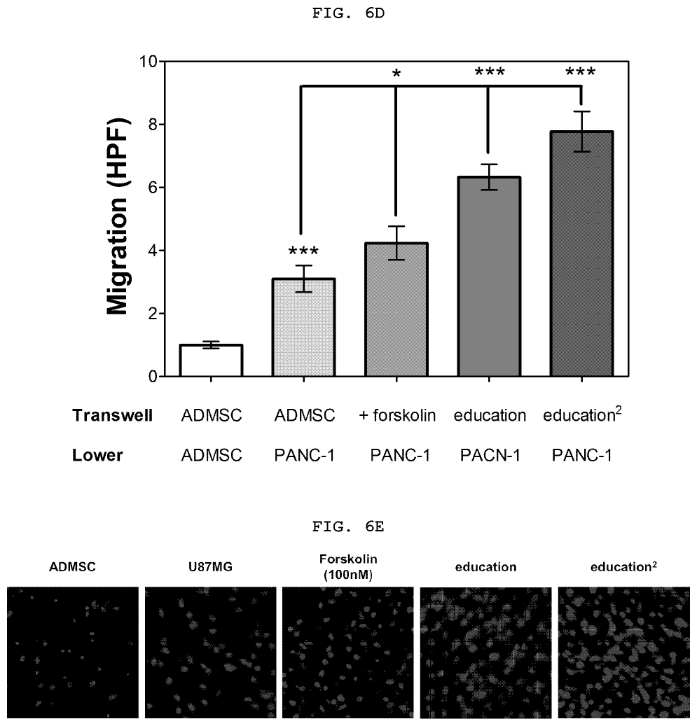

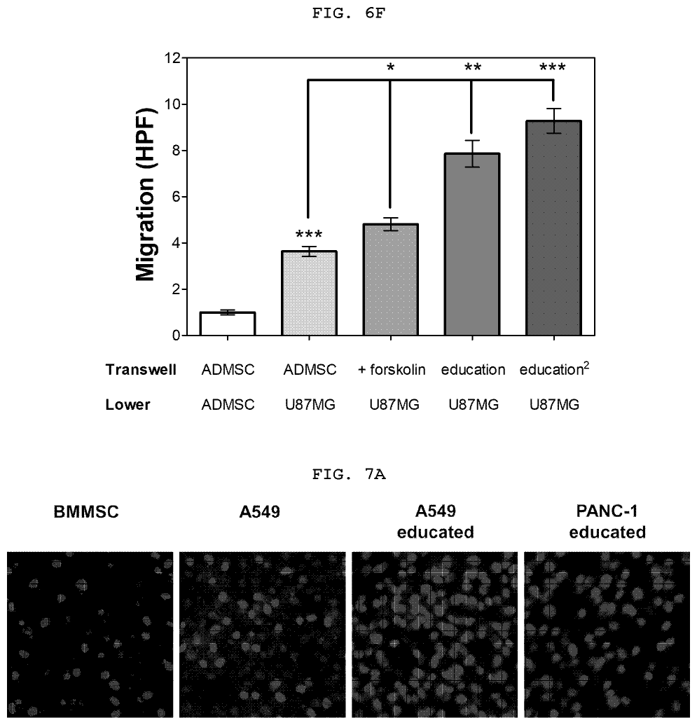

After the cultivation, the cells of the lower wells were fixed with methanol, and the nuclei of stem cells stained with DAPI (4′,6-diamidino-2-phenylindole). Subsequently, stem cells migrated into the lower wells were counted through a confocal fluorescence microscope in order to measure their mobility (FIGS. 6A to 6F). As shown in FIGS. 6A to 6F, when forskolin was treated to the stem cells, the ability of stem cells to migrate into cancer cells was significantly increased. The stem cells previously educated with cell-free culture medium of cancer cells showed better mobility than the only forskolin-treated stem cells. Further, the combined treated group (education² or e²) showed the best ability to migrate into cancer cells. This suggests that the education method of stem cells of the present invention can significantly improve the targeting ability of stem cells to cancer cells.

### 1-5: Investigation Whether Stem Cell Education is Cancer Cell-Specific

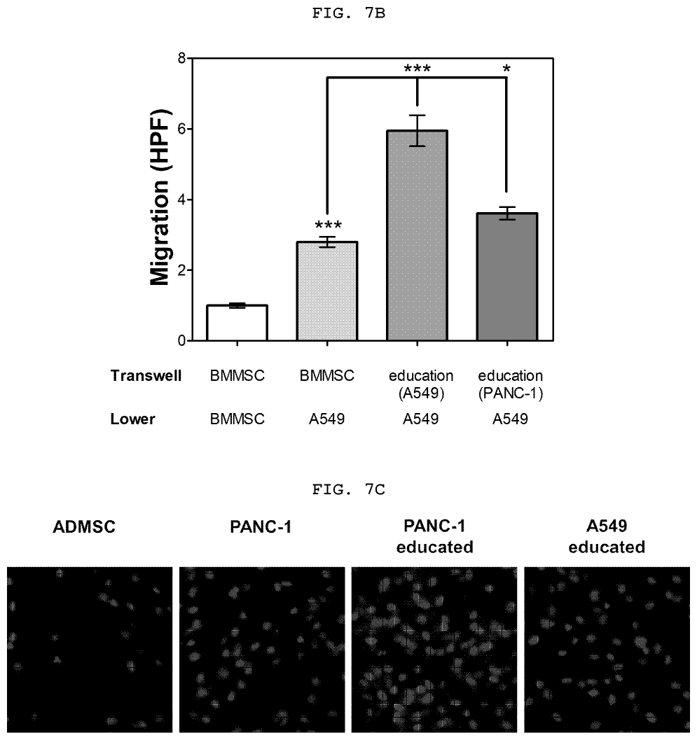

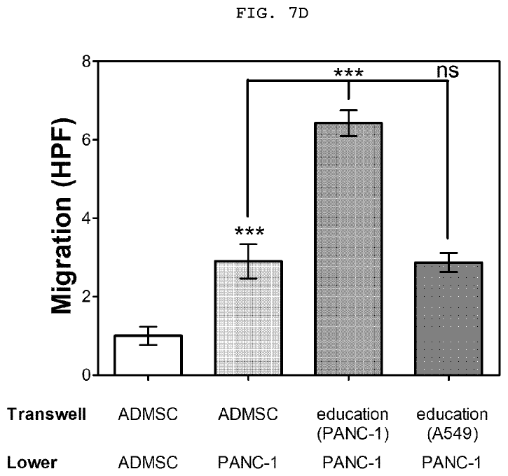

From the above results, the present inventors conducted the above except that the target cancer cells were changed in order to investigate whether the education of stem cells using culture medium of cancer cells specifically acts on the corresponding cancer cells or the same effect on other cancer cells. The similar experiment as in Example 1-4 was performed. Particularly, in the case of lung cancer cells, the present inventors dispense 5×10⁴ of bone marrow-derived mesenchymal stem cells (BMMSCs) as a control and lung cancer cells (A549 and H1975) as experimental groups were dispensed onto the lower well, respectively, and cultivated for 24 hours at 37° C. Simultaneously with the cultivation of the lower well, the BMMSCs were inoculated into a cell-free medium in which lung cancer cells (A549) had been cultured at 37° C. for 24 hours or cell-free medium in which pancreatic cancer cells (PANC-1) had been culture at 37° C. for 48 hours, and then cultured at 37° C. for 24 hours, followed by dispensing 5×10⁴ cells onto the transwells. The transwells were inserted into the lower wells and further cultured for 6 hours (education). In the case of pancreatic cancer cells, 5×10⁴ cells of adipose-derived mesenchymal stem cells (ADMSCs) as a control group and pancreatic cancer cells (PANC-1) as a control group were dispensed onto the lower wells and cultured at 37° C. for 48 hours. Simultaneously with the culture of the lower well, like the lung cancer cells, ADMSCs were inoculated into a cell-free medium in which lung cancer cells (A549) had been cultured at 37° C. for 24 hours or a cell-free medium in which pancreatic cancer cells (PANC-1) had been culture at 37° C. for 48 hours, and then cultured at 37° C. for 24 hours, followed by dispensing 5×10⁴ cells in the transwells. The transwells were inserted to the lower wells and the cells were further cultivated for 3 hours (education). As a result, as shown in FIGS. 7a to 7d, the stem cells educated different types of cancer cells showed a mobility that does not significantly differ from the control stem cells. This suggests that the education of stem cells of the present invention is specific to targeted cancer cells.

### Example 2: Measurement of Stem Cell In Vivo Cancer-Targeting Ability

BALB/c nude mice were used to measure the in vivo mobility of stem cells according to an embodiment of the present invention. The tumor model mice was prepared by inoculating cancer cells (lung cancer cell: A549; brain cancer cell: U87MG; and pancreatic cancer cell: PANC-1) to stimulate stem cells, and after stem cells were injected, the stem cells in the tumor tissue were counted in order to evaluate their mobility to tumor tissue.

### 2-1: Analysis of Tumor Cell-Targeting Ability in Lung Cancer Model

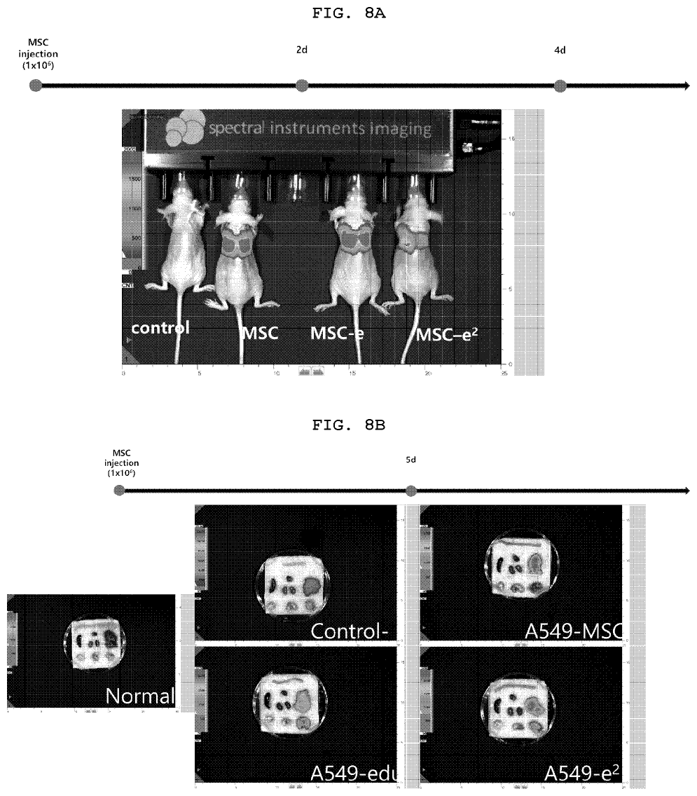

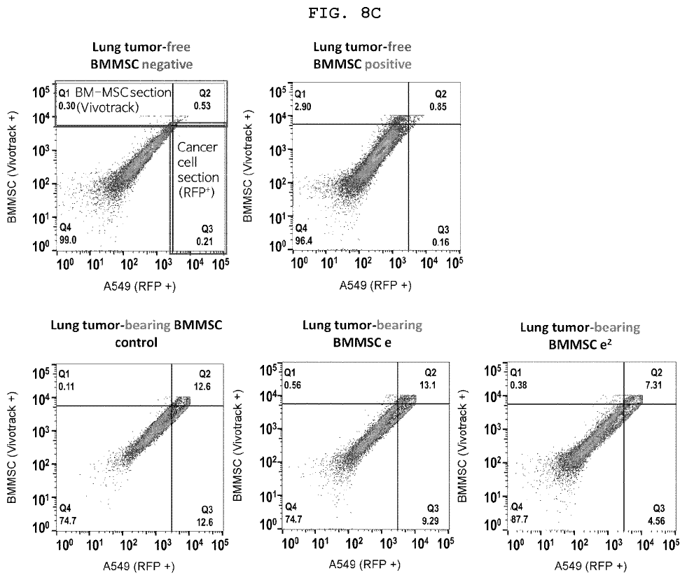

Particularly, lung cancer models were generated by inoculating BALB/c mice with 1×10⁶ lung cancer cells (A549-luciferase-RFP) genetically engineered to express fluorescent proteins and a bioluminescence enzyme (luciferase). After 4 to 8 weeks, after confirming that the lung cancer was generated by analyzing the size of the lung cancer using in vivo imaging system (IVIS) (FIG. 8A), 1×10⁶ cells of fluorescently labeled (vivotrack 680) bone marrow-derived stem cells (BMMSCs) were injected intravenously through the tail vein of the lung cancer model mice. After 5 days from the intravenous injection, the distribution of stem cells was investigated by ex vivo imaging after excising major organs including tumors tissue after sacrificing the experimental animals (FIG. 8B). In addition, the excised lung tissues containing lung cancer cells after imaging were single-celled and analyzed by flow cytometry in order to quantify the amount of stem cells that penetrated into the tumor tissue. The tissues excised for the single-celled process were chopped and incubated at 37° C. for 30 minutes with collagen degrading enzyme. The cultured tissues were filtered through a filter having a pore-size of 10 μm, and the filtered cells were washed with PBS. After resuspending the separated cells in 5% FBS/PBS aqueous solution, cell distribution of each experimental group was measured by performing flow cytometry analysis according to the emission wavelength of RFP and vivotrack 680 (FIG. 8C).

As a result, as shown in FIGS. 8A to 8C, it was confirmed that the target ability of stem cells to tumor tissues was improved when the two education methods according to an embodiment of the present invention were applied. In FIG. 8c, it can be interpreted that the first quadrant (Q1) and the second quadrant (Q2) are regions where stem cells are present, and the second quadrant (Q2) and third quadrants (Q3) are regions where cancer cells are present. The forth quadrant (Q4) is the area where normal cells are present in tumor tissue. From the results of the flow cytometry analysis of FIG. 8C, cells present in the second quadrant (Q2) (a section in which cancer cells and stem cells are combined) are increased by educated stem cell treatment, and the ratio of Q2/Q3 in education was increased significantly compared to the control mesenchymal stem cells. In particular, in the experimental group (MSC-e²) educated by treating forskolin and cancer cell culture medium to the stem cells, although the percentage of cells present in the second quadrant (Q2) is rather low, this is because the proportion of cancer cells is relatively low, and the Q2/Q3 ratio is the highest, and it was confirmed that the target ability to cancer cells significantly increased by the education. This suggests that stem cells educated according to an embodiment of the present invention can be used very efficiently for drug delivery specific to cancer cells.

### 3-2: Analysis of Tumor Cell-Targeting Ability of Stem Cells in Pancreatic Cancer Model

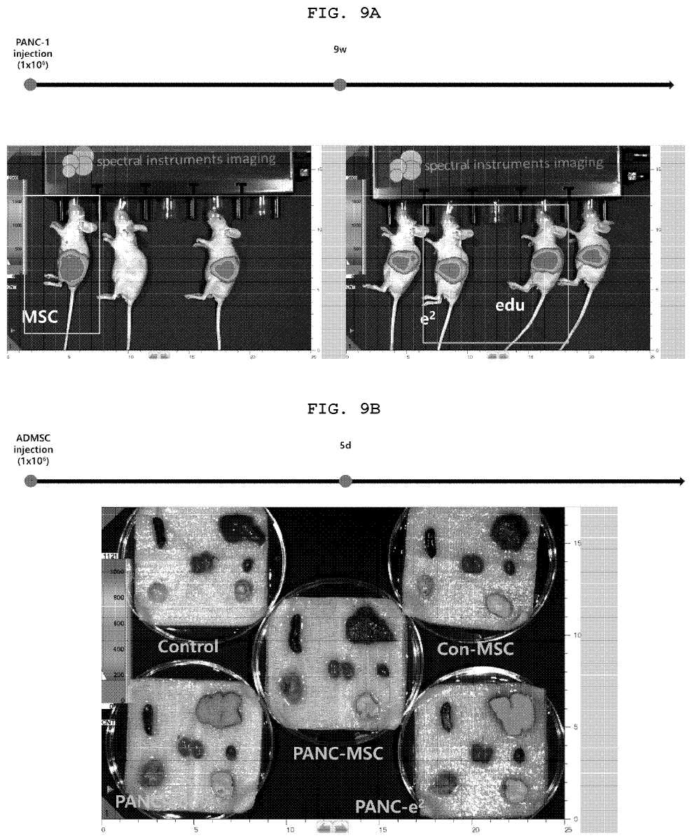

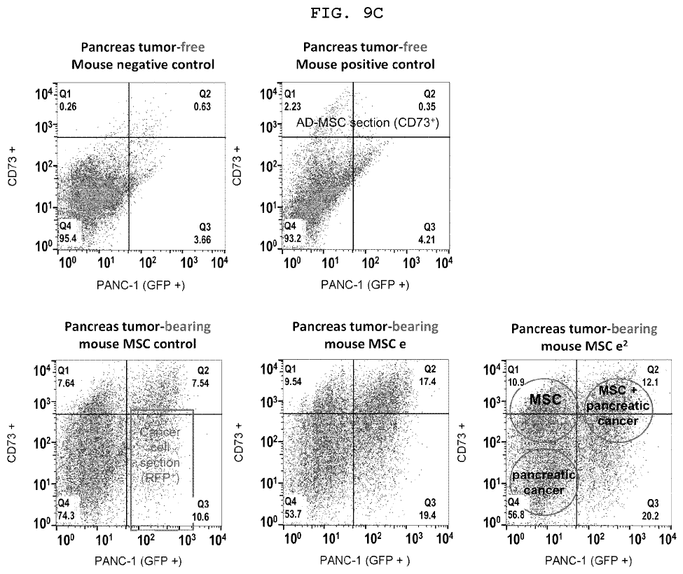

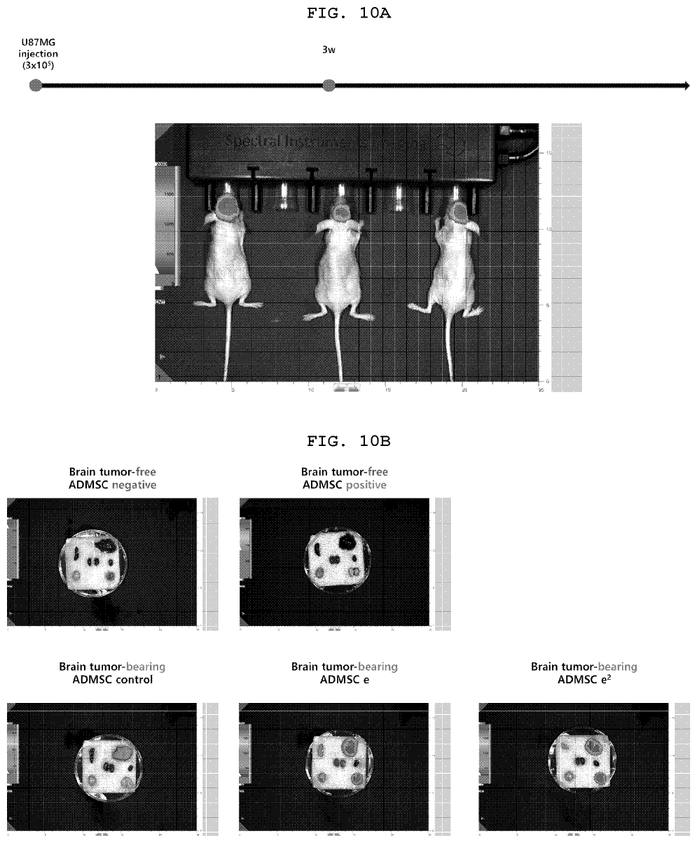

Particularly, pancreatic cancer model animals were generated by inoculating BALB/c nude mice with 1×10⁶ pancreatic cancer cells (PANC-1-luciferase-RFP) genetically engineered to express fluorescent proteins and luciferase. After 4 to 8 weeks, after confirming that the lung cancer was generated by analyzing the size of the lung cancer using in vivo imaging system (IVIS) (FIG. 9A), 1×10⁶ cells of fluorescently labeled (vivotrack 680) adipose-derived stem cells (ADMSCs) were injected intravenously through the tail vein of the pancreatic cancer model mice. After 5 days from the intravenous injection, the distribution of stem cells was investigated by ex vivo imaging after excising major organs including tumors tissue after sacrificing the experimental animals (FIG. 9B). Flow cytometry analysis was performed as described in Example 3-1 (FIG. 9C). As a result, as shown in FIGS. 9a to 9c, it was confirmed that the targeting ability of stem cells to tumor tissues was improved when applying the two education methods according to one embodiment of the present invention. Especially although the experimental group educated with forskolin and cancer cell culture medium (PANC-e²) showed relative low ratio of stem cells in Q1 and Q2 compared with the experimental group educated with cancer cell culture medium alone (PANC-e¹), it was confirmed that the targeting ability of the PANC-e² group to cancer cells was increased significantly from the finding that three types of cell populations which are normal pancreatic cells, mesenchymal stem cells (MSCs) and combined form of MSCs and pancreatic cancer cells were distinguished clearly. This suggests that stem cells educated according to one embodiment of the present invention can be used very effectively for drug delivery specific to cancer cells, particularly drug delivery for pancreatic cancer, which is a representative refractory malignant tumor.

### 3-3: Analysis of Tumor Cell Targeting Ability of Stem Cells in Brain Tumor Model

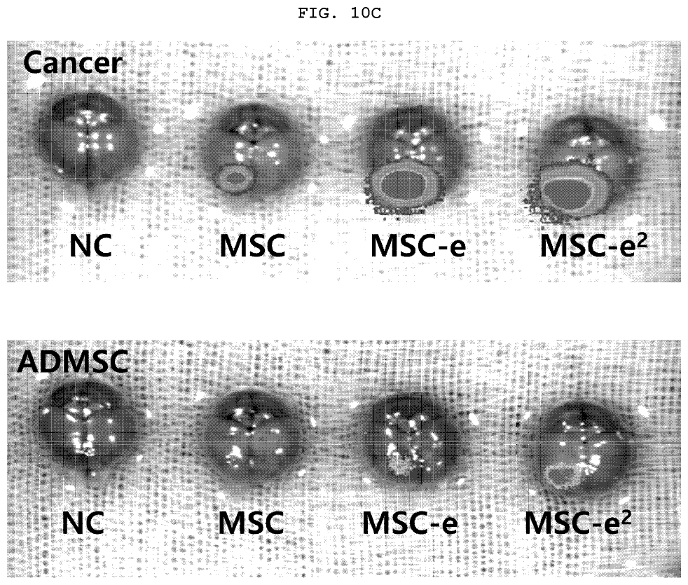

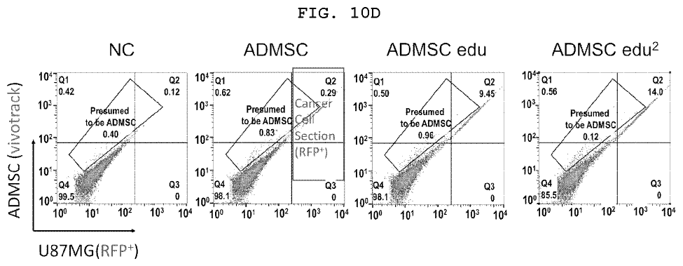

The brain tumor model animals were generated by inoculating BALB/c nude mice with 3×10⁵ brain tumor cells (U87MG-luciferase-RFP) genetically engineered to express fluorescent proteins and luciferase, by injecting the brain tumor cells into the brain through surgery. After confirming the size of the brain tumor by using an in vivo imaging system (IVIS) (FIG. 10A), 1×10⁶ adipose-derived stem cells (ADMSCs) fluorescently labeled (vivotrack 680) were injected intravenously through the tail vein of the brain tumor model mice. After 5 days form the intravenous injection, whole body in vivo imaging was performed on the experimental animals, and after sacrifice of the experimental animals, the main organs including the brain where the tumor is formed are excised and ex vivo imaging was performed in order to analyze the distribution of stem cells in organs and brain (FIG. 10B). Flow cytometry analysis was performed as described in Example 3-1 (FIG. 10C).

As a result, as shown in FIGS. 10A to 10D, stem cells educated according to an embodiment of the present invention exhibited a specific targeting ability to brain tumor, and in particular, as shown in FIG. 10D, shifts to the second quadrant (Q2) in the experimental group educated with brain tumor cell culture medium alone (ADMSC e) and the experimental group educated with the brain tumor cell culture medium and forskolin (ADMSC e2) was remarkable, and the targeting ability to brain tumor was significantly increased compared with the control group using uneducated stem cells. In particular, the results of this experiment were achieved by injecting stem cells into an experimental animal through intravenous injection, and proved that the stem cells accurately migrated to the brain tumor by crossing the blood-brain barrier (BBB) and it provides a clue to solve the important technical problems in the treatment of brain tumors in which chemotherapy has been limited due to the presence of the blood-brain barrier.

The present invention has been described with reference to the above-described embodiments, but these are merely exemplary, and those skilled in the art will understand that various modifications and equivalent other embodiments and experimental examples are possible therefrom. Therefore, the true technical protection scope of the present invention should be determined by the technical spirit of the appended claims.

1. A method of improving the migration ability of stem cells into cancer cells comprising:
educating the stem cells by treating the stem cells with a cell culture medium of in vitro cell culture of cancer cells obtained from a cancer patient to be treated and optionally an anion channel activator.

2. The method of claim 1, wherein the stem cells are embryonic stem cells or mesenchymal stem cells.

3. The method of claim 2, wherein the mesenchymal stem cells are bone marrow-derived mesenchymal stem cells, adipose-derived mesenchymal stem cells, umbilical cord-derived mesenchymal stem cells, dental pulp-derived stem cells or peripheral blood-derived stem cells.

4. The method of claim 1, wherein the anion channel activator is Cl⁻ channel activator or bicarbonate channel activator.

5. The method of claim 1, wherein the anion channel activator is forskolin, denufosol, brevenal, lubiprostone, N-aroylaminothiazole “activators” (E_{act}) analogue.

6. A composition comprising an anion channel activator and a cell culture medium of in vitro cell culture of cancer cells obtained from a cancer patient to be treated as an active ingredient.

7. The composition of claim 6, wherein the stem cells are embryonic stem cells or mesenchymal stem cells.

8. The composition of claim 7, wherein the mesenchymal stem cells are bone marrow-derived mesenchymal stem cells, adipose-derived mesenchymal stem cells, umbilical cord-derived mesenchymal stem cells, dental pulp-derived stem cells or peripheral blood-derived stem cells.

9. The composition of claim 6, wherein the anion channel activator is a Cl⁻ channel activator or a bicarbonate channel activator.

10. The composition of claim 6, wherein the anion channel is forskolin, denufosol, brevenal, lubiprostone, N-aroylaminothiazole “activators” (E_{act}) analogue.

11. A drug delivery composition for delivering anti-cancer drugs selectively to cancer cells comprising stem cells educated by treating with the composition of claim 6.

12. A stem cell-nano anti-cancer drug complex in which a carbon nanotube (CNT) or gold nanoparticle (AuNP) loaded with an anti-cancer drug is coupled to the surface of a stem cell, wherein the stem cell is educated by treating with the composition of claim 6.

13. The stem cell-nano anti-cancer drug complex of claim 12, wherein the anticancer drug is a chemical agent or a biologic agent.

14. The stem cell-nano anti-cancer drug complex of claim 13, wherein the chemical agent is doxorubicin, paclitaxel, ABT737, 5-fluorouracil, BCNU, CCNU, 6-mercaptopurine, nitrogen Mustard, cyclophosphamide, vincristine, vinblastine, cisplatin, mesotrexate, cytarabine thiotepa, busulfan or procarbazine.

15. The stem cell-nano anti-cancer drug complex of claim 13, wherein the biologic agent is immune checkpoint inhibitor, immune-activating protein or an antibody or a functional fragment thereof targeting a cancer marker protein.

16. The stem cell-nano anti-cancer drug complex of claim 12, wherein the nanoparticle is an inorganic nanoparticle or organic nanoparticle.

17. The stem cell-nano anti-cancer drug complex of claim 12, wherein the nanoparticle is coupled to the stem cell through an antibody specific for a stem cell marker protein, wherein the antibody is attached on the surface of the nanoparticle.

18. A pharmaceutical composition comprising the stem cell-nano anti-cancer drug complex according to claim 12 as an active ingredient.

19. A method of treating cancer in a subject in need of treatment comprising administering a stem cell-nano anti-cancer drug complex of claim 12.

20. A method of treating cancer in a subject in need of treatment comprising:
preparing an educated stem cell by educating a stem cell, wherein the educating is performed by treating the stem cell with cell culture medium of in vitro cell culture of cancer cells obtained from the subject and optionally an anion channel activator to the stem cells;
preparing an educated stem cell-nano anti-cancer drug complex by attaching a nanoparticle loaded with an anti-cancer drug to the educated stem cell; and
administering therapeutically effective amount of the educated stem cell-nano anti-cancer drug complex to the subject.
